(19) United States
(12) Patent Application Publication
(10) Pub. No.: US 2017/0224916 A1
(43) Pub. Date: Aug. 10, 2017

(54) FILTERS FOR INFUSION SETS

(71) Applicant: Novartis AG, Lichstrasse (CH)

(72) Inventors: Marc Sutter, Riehen (CH); Thomas Holbro, Basel (CH); Ahmed Besheer, Grenzach-Wyhlen (DE); Michael Billington, Riehen (CH)

(21) Appl. No.: 15/115,736

(22) PCT Filed: Jan. 30, 2015
(86) PCT No.: PCT/IB2015/050737

§ 371 Date: Aug. 1, 2016

Related U.S. Application Data
(60) Provisional application No. 61/935,014, filed on Feb. 3, 2014.

(51) Int. Cl. A61M 5/165; A61K 9/00; A61K 9/08; A61J 1/10; A61K 38/22

(52) U.S. Cl. CPC A61M 5/165; A61J 1/10; A61K 38/2221; A61K 9/08; A61K 9/0019; A61M 2205/7563

(57) ABSTRACT

In an acute care setting, rapid onset of the therapeutic effects of medication is highly desirable. The invention provides in-line filters suitable for rapid delivery of a positively charged protein therapeutic via intravenous administration.

### FIELD

The present invention relates to filters for use in infusion sets and methods of their use in administering protein therapeutics.

### BACKGROUND

Inline filters are used in intravenous therapy to trap particulates and ensure the sterility of the administered drug. A pore size of about 0.2 microns, e.g., 0.24 μm, is standard for preventing microbial contamination. Positively charged filters (sometimes referred to as endotoxin filters) may be chosen for use in infusion kits that administer positively charged protein therapeutics because the positive charge of the membrane repels the protein, minimizing adsorption of the protein to the filter. Adsorption of the protein to the filter is undesirable because the protein attached to the filter does not reach the patient, causing a reduction in the effectively administered dose. In an acute care setting, the benefits of rapidly delivering effective intravenous medication are well-recognized in the medical field and adsorption is subject to regulatory control.

### BRIEF SUMMARY

The inventors tested positively charged and neutral inline filters with a pore size of about 0.2 microns and discovered that only certain filters were suitable for infusing a positively charged protein therapeutic. This discovery was unexpected, in view of the known properties of the filters. Experiments were performed using normal saline (0.9% NaCl) and 5% dextrose (5% glucose) as solvents.

### BRIEF DESCRIPTION OF THE DRAWINGS

FIG. 1 depicts an initial screening of the adsorption of the protein therapeutic H2 relaxin to filters from B. Braun. The x axis depicts the flushing volume and the y axis depicts the concentration of H2 relaxin in the sample. H2 relaxin was diluted into a 250 mL infusion bag containing 5% dextrose to a concentration of five micrograms per milliliter (mL). The first bar in each set of four bars depicts the concentration of H2 relaxin when flushed through an infusion line without a filter. The second and third bars depict the addition of a B. Braun Sterifix filter (4184637 and 4099303 respectively) and demonstrate that adsorption was observed up to about 25 milliliters of flushing volume. The fourth bar depicts the addition of a B. Braun Intrapur Plus filter (4183916) and demonstrates that adsorption was observed up to about 20 milliliters of flushing volume.

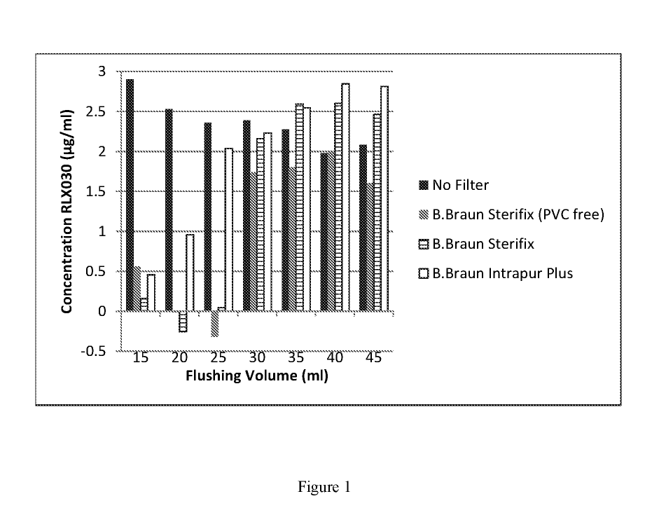

FIG. 2 depicts an initial screening of the adsorption of H2 relaxin to the B. Braun Perifix 4515501, the Pall Posidyne ELD (ELD96LLCE), the Pall Supor AEF (AEF1E) and the Alaris Impromediform MFX1826 filters. The x axis depicts the flushing volume and the y axis depicts the concentration of H2 relaxin in the sample. H2 relaxin was diluted into a 250 mL infusion bag containing 5% dextrose to a concentration of five micrograms per milliliter (mL). The first bar in each set of five bars depicts the concentration of H2 relaxin when flushed through an infusion line without a filter. The second, third, fourth and fifth bars depict the addition of a B. Braun Perifix, Pall Posidyne ELD Pall Supor AEF, and Alaris Impromediform MFX1826 filter respectively. No adsorption to the B. Braun Perifix or the Pall Posidyne ELD filters was observed. Adsorption to the Pall Supor AEF filter was observed up to about 15 mL of flush volume. Adsorption to the Alaris Impromediform MFX1826 filter was observed up to about 30 mL of flush volume.

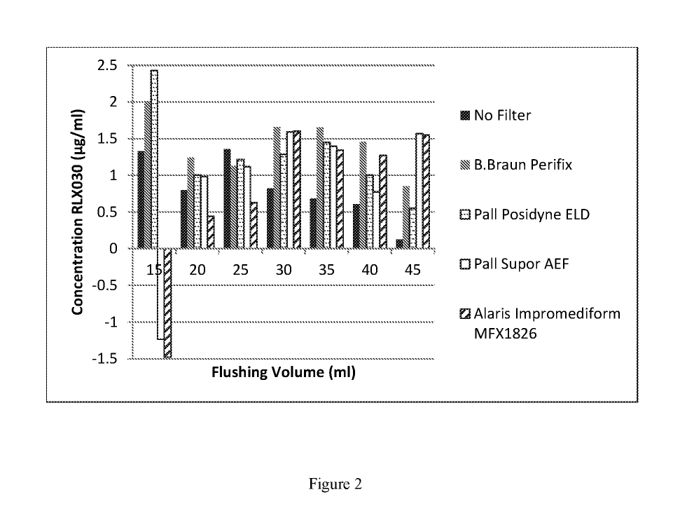

FIG. 3 depicts an initial screening of the adsorption of H2 relaxin to the Hospira Life Shield (12689-28), the RoweFil 120 nylon (A-2356) and the Terumo Extension Set (TF-SW231H). The x axis depicts the flushing volume and the y axis depicts the concentration of H2 relaxin in the sample. H2 relaxin was diluted into a 250 mL infusion bag containing 5% dextrose to a concentration of five micrograms per milliliter. The first bar in each set of four bars depicts the concentration of H2 relaxin when flushed through an infusion line without a filter. The second, third and fourth bars depict the addition of a Hospira Life Shield, RoweFil 120 nylon and Terumo Extension Set respectively. No adsorption to the RoweFil 120 nylon or the Terumo TF-SW231H was observed. Adsorption to the Hospira Life Shield filter was observed up to about 25 mL of flush volume.

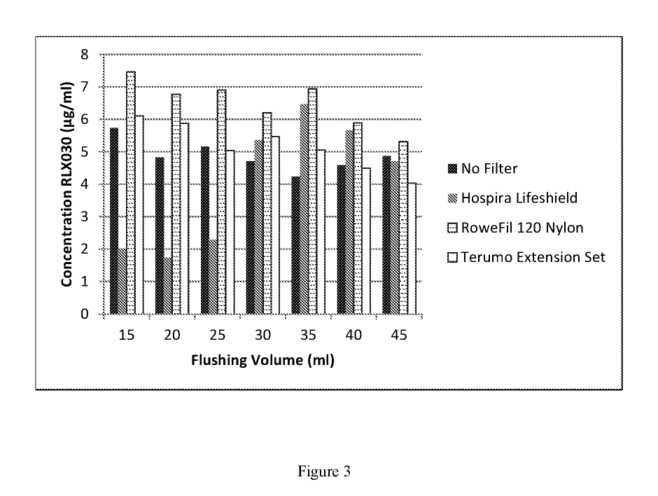

### DETAILED DESCRIPTION

### General Overview

The adsorption of a positively charged protein therapeutic to various filters was assessed by determining the volume of infusion solution passed through the filter before the protein concentration of the flow-through corresponded to the expected concentration. When this equilibration is achieved, the filter has reached its maximum protein adsorption. Thus, if a large flush volume is required to reach equilibrium, more protein is attaching to the filter. Conversely, a small flush volume indicates that the filter adsorbs the protein minimally, if at all, thus the protein therapeutic reaches the patient sooner.

### EMBODIMENTS OF THE INVENTION

The disclosure provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H. The invention includes all product codes of an infusion set when the filter is the same as the disclosed filter but other components of the infusion set, e.g., infusion lines, valves or needles may differ.

The disclosure provides a method of administering a positively charged protein therapeutic with a peripheral intravenous line comprising a 0.2 micron in-line intravenous filter wherein the filter is a Baxter 0.2 micron high pressure extended life filter.

The disclosure provides a method of administering a positively charged protein therapeutic with a peripheral intravenous line comprising a 0.2 micron in-line intravenous filter wherein the filter is a B. Braun Perifix.

The disclosure provides a method of administering a positively charged protein therapeutic with a peripheral intravenous line comprising a 0.2 micron in-line intravenous filter wherein the filter is a Codan IV STAR Plus 5.

The disclosure provides a method of administering a positively charged protein therapeutic with a peripheral intravenous line comprising a 0.2 micron in-line intravenous filter wherein the filter is a Pall Nanodyne ELD.

The disclosure provides a method of administering a positively charged protein therapeutic with a peripheral intravenous line comprising a 0.2 micron in-line intravenous filter wherein the filter is a Pall Posidyne ELD.

The disclosure provides a method of administering a positively charged protein therapeutic with a peripheral intravenous line comprising a 0.2 micron in-line intravenous filter wherein the filter is a Rowe RoweFil 120 Nylon.

The disclosure provides a method of administering a positively charged protein therapeutic with a peripheral intravenous line comprising a neutral-line intravenous filter wherein the filter is a Terumo TF-SW231H.

The disclosure also provides a method of administering a positively charged protein therapeutic with a peripheral intravenous line comprising a 0.2 micron in-line intravenous filter wherein the filter is chosen from Baxter 0.2 micron high pressure extended life filter (e.g., 2C8671 and 2H5660), B. Braun Perifix (e.g., 451550), Codan IV STAR Plus 5 (e.g., 76.3402), Pall Posidyne/Nanodyne ELD (e.g., ELD96LL, ELD96LLCE, ELD96LYL, ELD96LLC), Rowe RoweFil 120 Nylon (e.g., A-2356), Terumo TF-SW231H and the protein therapeutic is present in an infusion bag containing sterile dextrose or sterile saline solution.

The disclosure further provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H, wherein the infusion line and in-line filter are flushed with up to about 10 mL of the protein therapeutic solution from the intravenous bag.

The disclosure further provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H, wherein the infusion line and in-line filter are flushed with up to about 15 mL of the protein therapeutic solution from the intravenous bag.

The disclosure still further provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H wherein the infusion line and in-line filter are flushed with up to about 20 mL of the protein therapeutic solution from the intravenous bag.

The disclosure provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H wherein the infusion line and in-line filter are flushed with up to about 30 mL of the protein therapeutic solution from the intravenous bag.

The disclosure also provides a method of administering a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H wherein the positively charged protein therapeutic is H2 relaxin.

The disclosure further provides a method of preparing an infusion set for a positively charged protein therapeutic with a peripheral intravenous line comprising a 0.2 micron in-line intravenous filter wherein the filter is chosen from a Baxter 0.2 micron high pressure extended life filter (e.g., 2C8671 and 2H5660), B. Braun Perifix (e.g., 451550), Codan IV STAR Plus 5 (e.g., 76.3402), Pall Nanodyne ELD (e.g., ELD96LLCE), Pall Posidyne ELD (e.g., ELD96LL, ELD96LYL and ELD96LLC), Rowe RoweFil 120 Nylon (e.g., A-2356) and Terumo Extension Set TF-SW231H.

In an embodiment, an excipient is added to the sample containers used to hold the analytical samples obtained from flushing the filters. The excipient prevents adsorption of the positively charged protein to the sample container. Adsorption of the protein to the sample container would erroneously be attributed to adsorption of the protein to the filter. Any excipients known in the art to be useful for this purpose can be used. Such excipients are well known and include by way of example, amphiphilic substances such as surfactants, e.g., polysorbate 20 and proteins, e.g., bovine serum albumin.

In an embodiment, prior to filter testing, the infusion bags were stored at room temperature and laboratory light for 30 hours to simulate the time of patient infusion. No change in concentration was observed during this time.

In an embodiment, H2 relaxin is a protein with a molecular weight from 5.4 to 6.4 kilodaltons, an isoelectric point of 7.8 to 8.8 and a net charge of +3.3 to +4.3 at pH 6. The protein keeps its net positive charge when dissolved in 5% dextrose or 0.9% NaCl.

### Definitions

The terms used herein have their ordinary meanings, as set forth below, and can be further understood in the context of the specification.

A “positively charged protein therapeutic” is a protein or peptide used for the prevention, amelioration or treatment of a disease or disorder. It carries a positive charge in solutions having a pH compatible with therapeutic use, e.g., approximately pH 4-9, 4-8, 4-7 or 4-6.

“Adsorption” is the binding of molecules to a surface of a material without actual migration into the material.

As used herein, “H2 relaxin” is a positively charged protein therapeutic. It encompasses human isoform 2 (H2) preprorelaxin, prorelaxin, and relaxin, including H2 relaxin. It includes biologically active H2 relaxin from recombinant, synthetic or native sources as well as biologically active relaxin variants, such as amino acid sequence variants. The term further encompasses active agents with H2 relaxin-like activity, such as H2 relaxin agonists and/or H2 relaxin analogs and portions thereof that retain biological activity, including all agents that competitively displace bound H2 relaxin from a relaxin receptor. H2 relaxin, as used herein, can be made by any method known to those skilled in the art. Also encompassed is H2 relaxin modified to increase in vivo half-life, e.g., conjugated H2 relaxins, modifications of amino acids that are subject to cleavage by degrading enzymes, and the like. The term further encompasses H2 relaxins comprising A and B chains having N- and/or C-terminal truncations. Also included within the scope of the term are other insertions, substitutions, or deletions of one or more amino acid residues, glycosylation additions, organic and inorganic salts and covalently modified derivatives of H2 relaxin, H2 preprorelaxin and H2 prorelaxin. All such variations or alterations in the structure of the H2 relaxin molecule resulting in variants are included within the scope of this disclosure so long as the biological activity of the H2 relaxin is maintained. Variants of H2 relaxin having biological activity can be readily identified using assays known in the art.

### Analytical Methods

Protein concentrations can be measured by using any assay known in the art to evaluate adsorption to the surfaces of the sample containers. Reverse phase high performance liquid chromatography (RP-HPLC), fluorescence, bioassay and immunoassay are examples of suitable assays. Adsorption can also be measured using any assay known in the art, e.g., optical and spectroscopic techniques. Ellipsometry, surface plasmon resonance, scanning angle reflectometry, optical waveguide lightmode spectroscopy, circular dichroism spectropolarimetry, fluorescence spectroscopy, neutron reflectometry, quartz crystal microbalance methods and atomic force microscopy are some of the more commonly used methods.

### Protein Adsorption at Solid-Liquid Interfaces

Protein adsorption to solid surfaces such as filters is an inherently complex and unpredictable phenomenon, as many aspects of the characteristics of both the proteins and the surfaces are involved. Proteins are complex molecules possessing primary, secondary, tertiary and sometimes quaternary structures. Small changes in the environment can change the properties of a protein, e.g., its structure, stability or isoelectric point. For example, adsorption onto surfaces can trigger either a gain or a loss of secondary structure.

Adding to the complexity of proteins is the complexity of filter surfaces. Different materials, polymers and their modifications result in different protein adsorption properties. Both proteins and filter surfaces typically have a surface charge which can be gauged by zeta potential measurement. The attractive and repellant forces interact when proteins are adsorbed to filters and adsorption leads to a change in the zeta potential at the surface. Protein adsorption properties differ vastly and depend on many protein properties such as stability, isoelectric point, amino acid composition and surface charge as well as on filter properties such as hydrophobicity, charge, chemical structure and available surface area and, also, properties of the protein formulation such as pH, buffer, ionic strength and excipients.

### Infusion Filters

Infusion filters tested include the following. Characteristics of these filters and their suitability for use in H2 relaxin infusion are shown in Table 1.

Alaris Impromediform MFX1826 (Alaris, Lüdenscheid, Germany); B. Braun Intrapur Plus (B. Braun 4099800, Melsungen Germany); B. Braun Intrapur Plus (B. Braun 4183916, Melsungen Germany); B. Braun Perifix (B. Braun 4515501, Melsungen Germany); B. Braun Sterifix (B. Braun 4184637, Melsungen Germany); B. Braun Sterifix (B. Braun 4099303, Melsungen Germany); Baxter Extension Set (Baxter 2C8671, Deerfield Ill. US); Baxter Extension Set (Baxter 2H5660, Deerfield Ill. US); Codan I.V. STAR Plus 5 (Codan 76.3402, Lensahn Germany); Codan I.V. STAR Plus 10 (Codan 76.3400, Lensahn Germany); Fresenius Kabi Inufil (Fresenius Kabi 2909502, Bad Homburg Germany); Hospira LifeShield® Extension Set (Hospira 12698-28, Lake Forest Ill., US); Pall Supor AEF (Pall AEF1E, St. Columb Major, Cornwall UK); Pall Nanodyne ELD (Pall ELD96LLCE, St. Columb Major, Cornwall UK); Pall Posidyne ELD (Pall ELD96LL, St. Columb Major, Cornwall UK); Pall Posidyne ELD (Pall ELD96LLC, St. Columb Major, Cornwall UK); RoweFil 120 Nylon (RoweMed AG A-2356, Parchim Germany); and Terumo Terufusion Final Filter (Terumo TF-SW231H, Tokyo Japan).

TABLE 1
Characterization of Filters and Compatibility with H2 Relaxin Infusion

| | | | | | Substantial |
|---|---|---|---|---|---|
| | | Product | | | Adsorption of |
| Source | Filter Name | Code | Material | Charge | H2 Relaxin |
| Alaris | Impromediform | MFX1826 | PES | Positive | Yes |
| B. Braun | Intrapur Plus | 4099800 | PES | Positive | Yes |
| B. Braun | Intrapur Plus | 4183916 | PES | Positive | Yes |
| B. Braun | Perifix | 4515501 | | Neutral | No |
| B. Braun | Sterifix | 4184637 | PES | Neutral | Yes |
| B. Braun | Sterifix | 4099303 | PES | Neutral | Yes |
| Baxter | Extension Set | 2C8671 | PES | Neutral | No |
| Baxter | Extension Set | 2H5660 | PES | Neutral | No |
| Codan | I.V. STAR Plus | 76.3402 | PES | Positive | No |
| | 5 | | | | |
| Codan | I.V. STAR Plus | 76.3400 | PES | Positive | Yes |
| | 10 | | | | |
| Fresenius | Inufil | 2909502 | PES | Positive | Yes |
| Kabi | | | | | |
| Hospira | LifeShield ® | 12689-28 | PES | Neutral | Yes |
| | Extension Set | | | | |
| Pall | Supor AEF | AEF1E | PES | Neutral | Yes |
| Pall | Nanodyne ELD | ELD96LLCE | Nylon | Positive | No |
| Pall | Posidyne ELD | ELD96LL | Nylon | Positive | No |
| Pall | Posidyne ELD | ELD96LLC | Nylon | Positive | No |
| Rowe | RoweFil 120 | AG A-2356 | Nylon | Positive | No* |
| | Nylon | | | | |
| Terumo | Terufusion | TF-SW231H | PS | Neutral | No |
| | Final Filter | | | | |

PES: Polyether Sulfone
PS: Polysulfone
*In dextrose

As used in this specification and the appended claims, the singular forms “a,” “an,” and “the” include plural referents unless the content clearly dictates otherwise. Thus, for example, reference to “a protein” includes a mixture of two or more proteins, and reference to “the agent” includes reference to one or more agents and equivalents thereof known to those skilled in the art, and so forth.

It will be clear that the invention may be practiced otherwise than as particularly described in the foregoing description and examples. Numerous modifications and variations of the present invention are possible in light of the above teachings and, therefore, are within the scope of the appended claims.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claim.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention.

Further, all numbers expressing quantities of ingredients, reaction conditions, % purity, polypeptide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques.

### Example 1: Analytical Methods

For screening as described in the Brief Description of the Drawings, protein concentration was measured by protein fluorescence on a plate reader. In subsequent post-screening experiments, protein concentration was determined by the Quantikine Human Relaxin-2 Immunoassay (R&D Systems testing kit DRL200) (Sections 041, 043, and 044). Protein concentrations in the examples shown below were also measured by RP-HPLC measurements optimized by minimal adsorptive loss of the protein by choice of a suitable HPLC vial and by bracketing samples in the sequence with reference standards.

Bioactivity was determined using a cell-based cAMP production bioassay.

Adsorption of H2 relaxin to infusion bags and infusion lines containing either 5% dextrose or 0.9% saline was tested. Essentially no loss of H2 relaxin due to adsorption to the infusion bags or lines was observed at protein concentrations between 5 and 30 micrograms per milliliter following exposure for 0, 1 or 30 hours.

### Example 2: Serelaxin Adsorption to Filters in 0.9% NaCl

| | Material/ | Flush | % Recovery | |
|---|---|---|---|---|
| Filter | Charge | Aliquot | from Filter¹ | % Bioactivity² |
| Baxter Extension | PES | 5 mL | 92.6 | |
| Set 2C8671 | neutral | 10 mL | 95.3 | |
| | | 15 mL | 87.2 | |
| | | 20 mL | 89.9 | |
| | | 25 mL | 92.6 | |
| | | 30 mL | 100.8 | |
| | | 35 mL | 106.2 | |
| | | 40 mL | 117.1 | 84 |
| Baxter Extension | PES | 5 mL | 106.2 | |
| Set 2H5660 | neutral | 10 mL | 68.1 | |
| | | 15 mL | 98.1 | |
| | | 20 mL | 98.1 | |
| | | 25 mL | 95.3 | |
| | | 30 mL | 103.5 | |
| | | 35 mL | 117.1 | |
| | | 40 mL | 114.1 | 96 |
| Pall Posidyne | Nylon | 5 mL | 0.0 | |
| ELD ELD96LL | positive | 10 mL | 0.0 | |
| | | 15 mL | 35.4 | |
| | | 20 mL | 70.8 | |
| | | 25 mL | 81.7 | |
| | | 30 mL | 68.1 | |
| | | 35 mL | 81.7 | |
| | | 40 mL | 81.7 | 82 |

¹At a concentration of 5 μg/mL
²At a concentration of 30 μg/mL

Surprisingly, a positive charge on the filter did not predict whether it adsorbed the positively charged protein. Substantial differences in adsorption were observed when different positively charged filters were tested. For example, almost no adsorption to the filters in the neutral PES Baxter Extension Sets 2C8671 and 2H5660 were observed and a flushing volume of 20 mL was sufficient to reach equilibrium. Some adsorption to the Pall Posidyne ELD ELD96LL was observed. The results are shown above as Example 2.

### Example 3: Serelaxin Adsorption to Filters in 5% Dextrose

| | Material/ | Flush | % Recovery from |
|---|---|---|---|
| Filter | Charge | Aliquot | Line and Filter |
| Baxter Extension | PES | 15 mL | 65.2 |
| Set 2C8671 | neutral | 20 mL | 86.0 |
| Baxter Extension | PES | 15 mL | 68.6 |
| Set 2H5660 | neutral | 20 mL | 84.4 |
| Pall Posidyne | Nylon | 15 mL | 82.3 |
| ELD ELD96LL | positive | 20 mL | 93.6 |
| Codan I.V. Star | PES | 15 mL | 103 |
| Plus 5 76.3402 | positive | 20 mL | 100 |

In 5% dextrose, minimal or no adsorption to the neutral Baxter Extension Sets 2C8671 2H5660 occurred, requiring a flushing volume of only 15 mL. Also, minimal or no adsorption to the positively charged Pall Posidyne ELD 96LL and Codan I.V. STAR Plus 5 was observed. This is shown above as Example 3.

In 5% dextrose solution, H2 relaxin showed minimal or no adsorption to positively charged nylon filters. Both positively charged PES filters and neutral filters could show substantial adsorption or very little to no adsorption.

The experimental data revealed substantial differences of protein adsorption to different filters. For example, in 0.9% NaCl the Pall Posidyne ELD filter showed initial H2 relaxin protein adsorption and recovery values of >80% were reached after >20 mL flush volume. The RoweFil 120 Nylon filter showed less than 20% recovery even after >30 mL flush volume when tested in saline but had a favorable adsorption profile when tested in dextrose. In 5% dextrose, the RoweFil 120 Nylon filter, which strongly adsorbs H2 relaxin when using 0.9% NaCl infusion bags, did not substantially adsorb H2 relaxin. A flushing volume of 10 mL through the RoweFil120 Nylon filter was adequate when using 5% dextrose.

Surface (zeta) potential measurements of both the proteins and the tested filters can only partially explain some of the observed adsorption properties. For instance, the neutral Hospira LifeShield® PES filter, which adsorbed strongly in 5% glucose solution, turned out to bear a strong negative charge, explaining the adsorption of the positively charged proteins investigated. In saline solution, however, the large surplus of ions could lead to masking of the actual surface charge, thus resulting in a less negative total charge and thus less attraction for the positively charged proteins.

We claim:

1. A method of administering a positively charged protein therapeutic with a peripheral intravenous line comprising an in-line intravenous filter wherein the filter is chosen from a B. Braun Perifix, a Baxter 0.2 micron high pressure extended life filter, a Codan I.V. STAR Plus 5, a Pall Nanodyne ELD, a Pall Posidyne ELD, a RoweMed RoweFil 120 Nylon and a Terumo TF-SW231H.

2. The method of claim 1, wherein the filter is a B. Braun Perifix.

3. The method of claim 1, wherein the filter is a Baxter 0.2 micron high pressure extended life filter

4. The method of claim 3 wherein the Baxter 0.2 micron high pressure extended life filter is Baxter Extension Set 2C8671 or Baxter Extension Set 2H5660.

5. The method of claim 1, wherein the filter is a Codan I.V. STAR Plus 5.

6. The method of claim 5 wherein the Codan I.V. STAR Plus 5 is Codan 76.3402.

7. The method of claim 1, wherein the filter is a Pall Nanodyne ELD.

8. The method of claim 7 wherein the Pall Nanodyne ELD is a Pall Nanodyne ELD96LLCE.

9. The method of claim 1, wherein the filter is a Pall Posidyne ELD.

10. The method of claim 9 wherein the Pall Posidyne ELD is Pall Posidyne ELD96LL, Pall Posidyne ELD96LLC or Pall Posidyne ELD96LYL.

11. The method of claim 1, wherein the filter is a RoweMed RoweFil 120 Nylon.

12. The method of claim 11 wherein the RoweMed RoweFil 120 Nylon is a RoweMed AGA-2356.

13. The method of claim 1, wherein the filter is a Terumo TF-SW231H.

14. The method of claim 1, wherein the protein therapeutic is present in an infusion bag containing sterile dextrose or sterile saline solution.

15. The method of claim 1, wherein the infusion line and in-line filter are flushed with up to about 30 mL of the protein therapeutic solution from the intravenous bag.

16. The method of claim 15, wherein the infusion line and in-line filter are flushed with up to about 20 mL of the protein therapeutic solution from the intravenous bag.

17. The method of claim 16, wherein the infusion line and in-line filter are flushed with up to about 15 mL of the protein therapeutic solution from the intravenous bag.

18. The method of claim 17, wherein the infusion line and in-line filter are flushed with up to about 10 mL of the protein therapeutic solution from the intravenous bag.

19. The method of claim 1, wherein the positively charged protein therapeutic is H2 relaxin.

20. A method of preparing an infusion set for administering a positively charged protein therapeutic with a peripheral intravenous line comprising a neutral or positively charged in-line intravenous filter wherein the filter is chosen from a B. Braun Perifix, a Baxter 0.2 micron high pressure extended life filter, a Codan I.V. STAR Plus 5, a Pall Nanodyne ELD, a Pall Posidyne ELD, a RoweMed RoweFil 120 Nylon and a Terumo TF-SW231H.
